(19) United States
(12) Patent Application Publication
(10) Pub. No.: US 2009/0107866 A1
(43) Pub. Date: Apr. 30, 2009

(54) Rupturable bubble package

(76) Inventor: Michael Dunn-Rankin, Venice, FL (US)

Correspondence Address: Michael Dunn-Rankin, 502 Warwick Dr., Venice, FL, 34293, US

(21) Appl. No.: 11/978,361

(22) Filed: Oct. 30, 2007

(51) Int. Cl. B65D 83/04; A61B 19/02

(52) U.S. Cl. 206/363; 206/349; 206/438; 206/522; 206/528

(57) ABSTRACT

A ruptural bubble package for containing an implement such as, for example, a drinking straw, a toothpick or medical instruments, The bubble package is characterized by its construction from a flexible, substantially impervious material capable of maintaining a positive pressure of air or other gaseous fluids within the package once it is sealed. There are weakened portions or locations on the package that will burst open when an exterior finger pressure is applied to the outside of the package.

### BACKGROUND OF THE INVENTION

The present invention relates to a ruptural bubble package that contains a sanitized implement. While a first preferred embodiment will be described wherein the implement is a drinking straw, it is to be understood that the particular implement placed within the package is not to be considered a limiting factor on the scope of the invention. In fact, it is contemplated that the present invention would have utility for a variety of implements including, for example, even sterilized, medical or surgical instrument including the packaging of tooth picks.

The invention is characterized by its construction from a flexible, substantially impermeable to define an enclosure into which the implement is inserted and sealed therein. By “substantially impervious” is meant to include a material capable of maintaining a quantity of a gaseous fluid inside the enclosure, preferably a gas such as, for example, air or an inert sterilized gas, when the package is sealed. The package comprises a front panel, a back panel, and opposed top and bottom panels that are disposed in interconnecting relationships between the front and back panels to define the enclosure into which the implement is placed. A weakened location or portion is formed on at least one of the front, back, top and side panels such that by squeezing the sealed container between one's thumb and fingers will cause an increase of air or gas pressure within the sealed container resulting in a rupture of the weakened portion to thereby permit access to the implement. The increase in air pressure is most important.

### DESCRIPTION OF THE PRIOR ART

The prior art teaches a variety of what might be described as ruptural packages for numerous articles or implements. For example, common drinking straws are typically enclosed within a paper or plastic packages or sealed sleeves. The package is torn, or ruptured, or cut with a knife or scissors or even teeth have been used to open the package for its intended use. In similar fashion, bandages, both adhesive and gauze, are typically provided in a sterile container that is ruptured, or torn in the above noted fashion to obtain access to the bandage. Nowadays, literally everything from soup to nuts is often provided to the ultimate user in what may be described as a rupturable package. Dry soup mixes are provided in envelopes that are torn open, and nuts and bolts are frequently sold in plastic pouches.

Even some medications are provided to the ultimate user in packaging that is sometimes referred to as a bubble pack or bubble card wherein one surface is typically formed from a foil-type material through which the medication is punched to rupture the foil. Yet another example of what might be termed as a rupturable package is a sealed plastic film envelope containing a drinking straw that one finds attached to the outside of a single beverage container.

However, with all such packages known, one obtains access to the item contained therein either by tearing the package, cutting the package or opening a zipper-type closure formed on the package. While such packages are quite suitable for their intended use, there remains a need for a rupturable package that could simply be opened by squeezing the package to “pop” the package open and thereby gain access to the item placed inside. Of ancillary value, though nevertheless significant, is the fact that such a package would typically make a popping sound on being opened, and this sound could be an amusement to the user. On a more serious vein, such a package would have the further advantages of not requiring any external implements such as, for example, scissors, knifes, sharp fingernails or even a person's teeth, for opening, because such a package could be opened with one hand, and would not result in the creation of small package fragments which are often discarded as litter.

It should be understood that there are known packages that can be opened just be squeezing the same. Examples of such packages will be disclosed below.

US published Application No. 2003/0019781 discloses a capsule container system (CCS) which relates to a small, single use disposable container which contains substances or solutions therein with the capsule having easy opening capabilities. The opening can be obtained by simply squeezing the flexible container whereby the container will burst because of a weakened score line in the surface of the container.

U.S. Pat. No. 3,189,227 discloses a string of small flexible containers having a score or weakened line between the packages and when any one of the packages is torn from the string of the containers, a nozzle is exposed which will expel the fluid contained in the package by squeezing the package by the user's fingers.

Any one of the noted squeezable packages are quite different from the inventive concept disclosed and claimed herein as will be explained in the following paragraph below.

### BRIEF DESCRIPTION OF THE INVENTION

The present invention relates to a rupturable bubble package for containing in implement and wherein the package comprises an enclosure formed from a substantially impervious, flexible material, such as plastics, foil or paper. The enclosure comprises a front panel, a back panel, and opposed top and bottom panels which are disposed in an interconnecting relationship between the front and the back panels to define the enclosure. It is, of course understood that the terms front, back, top and bottom are relative to the orientation of the package, and are not to be taken as absolute terms with respect to the embodiments described in greater detail hereinafter. The package could be obtained through the process of extrusion whereby a plastic sleeve is obtained that may be sealed at both ends. It is also to be understood that the phrase “substantially impervious” is intended to convey that the material from which the enclosure is formed does not readily permit any fluid through the surface of the sealed package to pass there through. In its preferred embodiment, the fluid inside the sealed package is typically a gas or simply air. More particularly, when sealed, the package of this invention is capable of maintaining a positive pressure inside the package and when squeezed, the pressure will momentarily increase so that the package can rupture as will be described below.

Rupturing of this package is accomplished by its construction to further include a weakened portion or area formed on at least one of the front, back, top and bottom panels. The weakened portion may be score line at or near the top of the package. The inventive concept can also be realized when a plastic sleeve is extruded, the bottom may be sealed, while the top may remain in an overlap that may glued together whereby a weaker closures or seal is formed that is weaker than the bottom seal and may ruptured when pressure is applied to the package. The overlaps may take different shapes.

The inventive concept, accordingly, comprises an article of manufacture possessing the features, properties and the relation of elements which will be exemplified in the articles hereinafter described, and the scope of the invention will be indicated in the claims.

### BRIEF DESCRIPTION OF THE DRAWINGS

FIG. 1 is a perspective view of the prior art container having a packaged drinking straw attached thereto, with the carton shown in phantom;

FIG. 2 is a perspective view illustrating a first embodiment of the inventive rupturable bubble package attached to the carton of FIG. 1;

FIG. 3 is a front elevational view of the embodiment shown in FIG. 2;

FIG. 4 is a side elevational view of the embodiment shown in FIG. 2;

FIG. 5 is an illustration of how the embodiment of FIG. 2 can be removed from the carton;

FIG. 6 illustrates the use of one hand squeezing the embodiment of FIG. 2 after a removal from the carton;

FIG. 7 is a depiction of the package of FIG. 6 after “popping” open;

FIG. 8 shows the removal of the straw from the opened package;

FIG. 9 is a front view of just the inventive package;

FIG. 10 is a side view of just the inventive package;

FIG. 11 is a front view of a second embodiment of the inventive package;

FIG. 12 is a side view of the second embodiment of the package of FIG. 11;

FIG. 13 is a front view of a third embodiment of the inventive package;

FIG. 14 is a side view of the embodiment of FIG. 13;

FIG. 15 is a front view of the fourth embodiment of the inventive package;

FIG. 16 is a side view of the embodiment of FIG. 15;

FIG. 17 is a front view of the fifth embodiment of the inventive package;

FIG. 18 is side view of the embodiment of FIG. 17.

FIGS. 19 and 20 show a different inventive concept wherein instead of weakened score lines, the package is closed by a weak closure;

FIG. 21 illustrates a different embodiment of FIGS. 19 and 20;

FIG. 22 shows the bubble package of FIG. 21 after it popped open;

FIGS. 23 and 24 show a different embodiment of FIGS. 22 and 23;

FIG. 25 Illustrates a different embodiment of FIG. 21;

FIG. 26 shows a different construction of the bubble package;

FIG. 27 shows a different construction of the package of FIG. 26;

FIG. 28 shows still a different construction of the package of FIG. 26;

FIG. 29 shows a bubble package having a top and bottom seals;

FIG. 30 shows the structure of FIG. 29 having different types of top and bottom seals;

FIG. 31 shows a top seal having an air pocket therein;

FIG. 32 shows a top seal having two air pockets therein;

FIG. 33 shows a top seal having an air pocket in front of the seal;

FIG. 34 shows a top seal having a circular air pocket therein;

FIG. 35 shows a top seal having differently sized air pockets therein.

### DETAILED DESCRIPTION OF THE INVENTION

FIG. 1 illustrates a state of the art container, generally indicated as 34. The container 30 has a drinking straw 32 attached thereto on a front 40 of the container 34 by way of a package 30. To use the straw, one would remove the container 30 from the drink container 34 and would open the package 30 as by tearing or cutting. The straw 32 could then be removed from the package 30 and insert the straw into the opening 36 of the drink container 34.

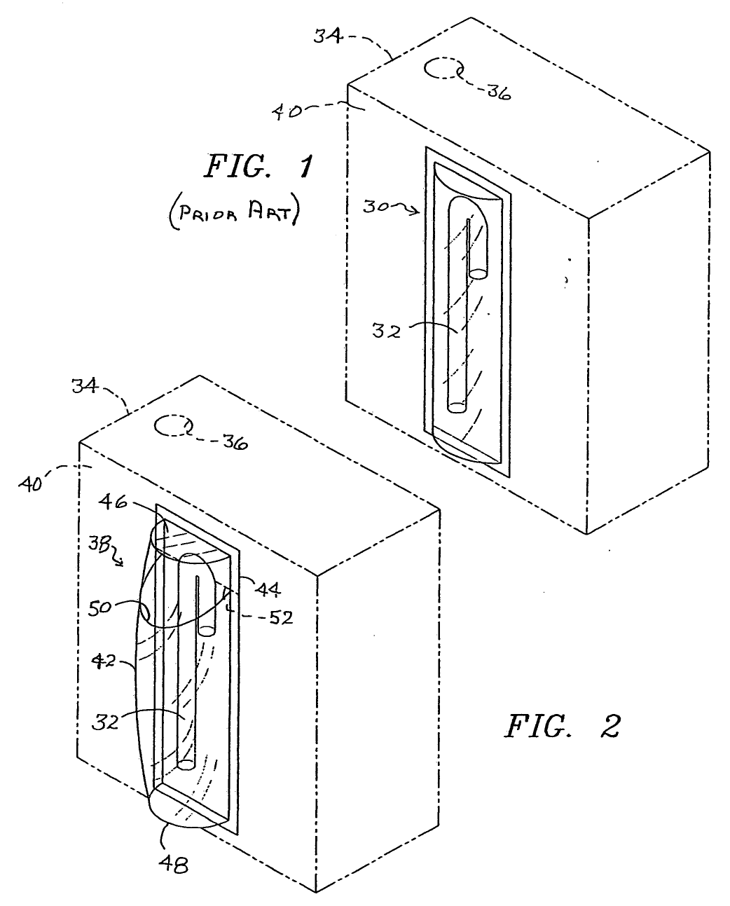

FIG. 2 illustrates the ruptural bubble package of the present invention. The ruptural package is generally indicated at 38. Reference characters assigned to previous Figs. will be carried over into subsequent Figs. While the package 38 is illustrated as being formed from a substantially clear or transparent material, it is to be understood that this is for illustrative purposes only. Ruptural package 38 is illustrated as being attached to a front 40 of a drink container 34 which also includes the aperture 36 for insertion of straw 32 once it is removed from the package 38. Bubble package 38 comprises an enclosure that is preferably formed from a flexible material, and the enclosure is defined by a front panel 42, a back panel 44, a top panel 46 and a bottom panel 48. As will be described in greater detail below, package 38 is preferably attached to the front 40 of the container 34 by a peelable adhesive between the back panel 44 and the front 40 of the container 34. FIG. 2 also shows a score line 50 in the material from which the bubble package is formed. Also shown in FIG. 2 is a hinge line 52 as more fully described below.

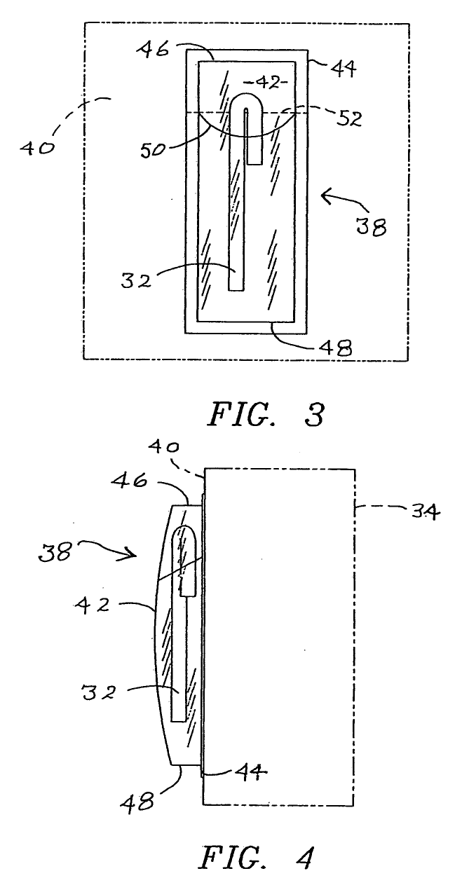

FIGS. 3 and 4 depict the embodiment of FIG. 2 in front and right side elevations, respectively.

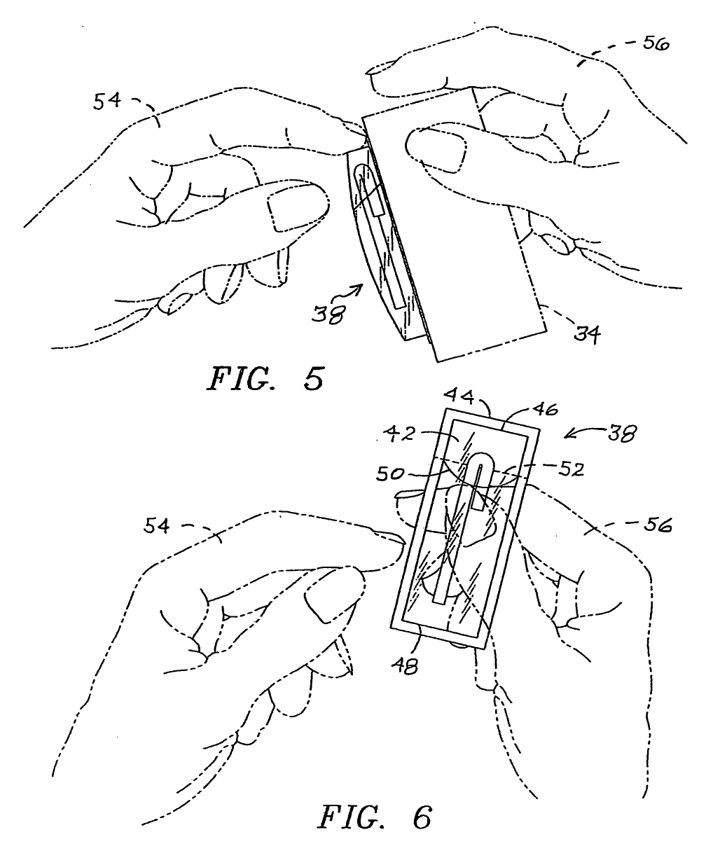

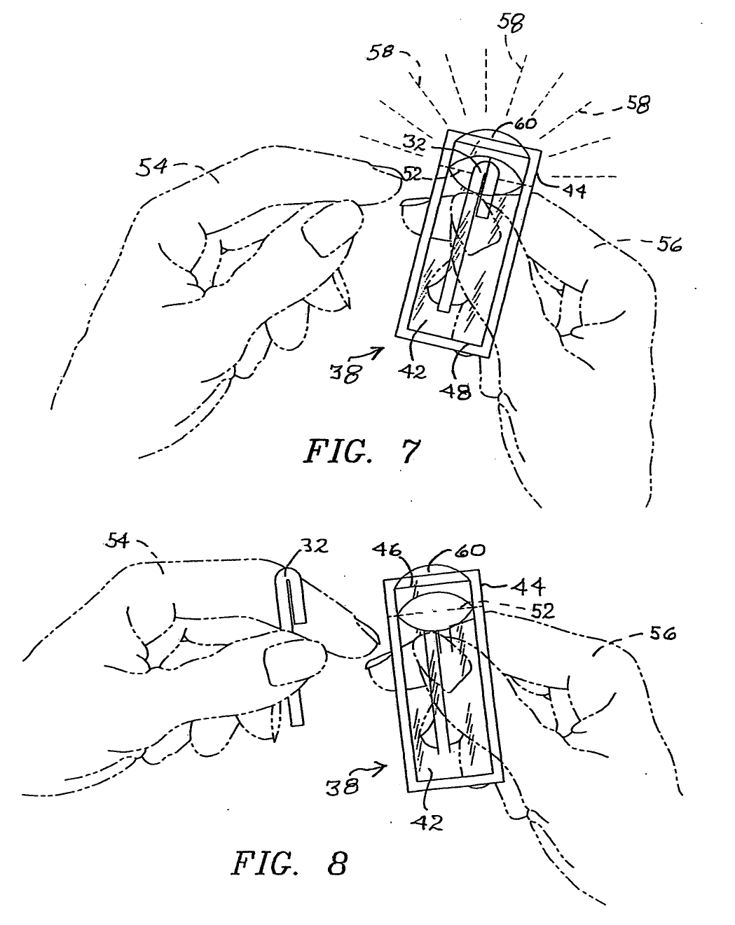

FIGS. 5-8 are perspective illustrations as to how the bubble packages 38 can be opened once they are detached from the basic drink carton 34. In Fig. the right hand 56 holds the drink carton while the left hand 54 pulls the bubble package 38 away from the carton 34. In FIG. 6 the right hand is squeezing the bubble package 38. Because the bubble package 38 is formed from a flexible and substantially impervious material that is capable of maintaining a positive pressure within the package 38. By squeezing the bubble package 38, the pressure of air within the package is temporarily increased which will cause the score line 50 to rupture. This rupture and escape of air is illustrated in FIG. 7 by the radial dashed lines 58. This rupture causes an upper segment 60 of the front panel 42 to spring upwardly to thereby provide access to the straw 32 inside the package 38. For further ease in removing the straw 32, the portion of the bubble package 38 above the hinge line 52 may actually be folded back. Upon squeezing rupturable bubble package 38, as depicted in the view of FIG. 6, one may experience an audible “pop” as the package 38 ruptures at the score line 50. Line 50 does not inhibit the integrity of the sealed package 38, but provide a weakened portion for rupturing the package 38 by the application of an outside pressure.

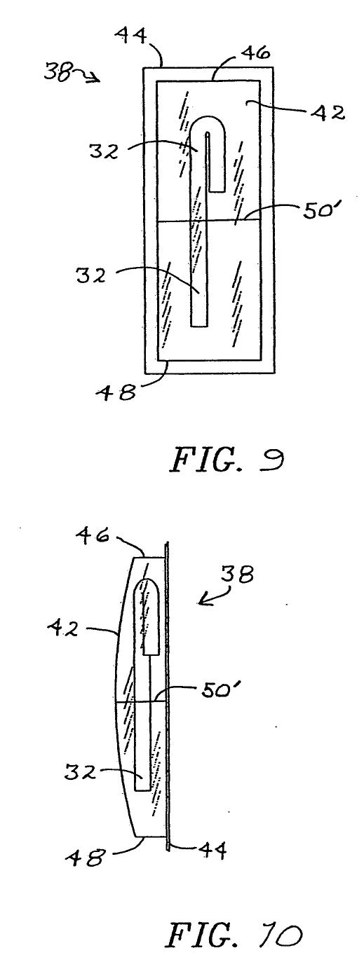

FIGS. 9 and 10 depict a second embodiment for the rupturable bubble package 38 wherein the weakened portion comprises the score line 50′ which is positioned substantially across the midpoint of front panel 42. Of course, in the views of FIGS. 9 and 10, as well as the remaining views provided, bubble package 38 is illustrated without being attached to any container. It is to be understood that the bubble package can be so constructed that it solely can contain any implements mentioned above. That is, the bubble package can contain a toothpick, just a straw, so-called toilettes, pills, food and other sanitary or hardware items that are hermetically sealed within the bubble package.

While score line 50′ is illustrated as being oriented horizontally across the front panel 42, it is to be appreciated that score line 50′ could also be oriented vertically, and the pressure applied would still cause the package 38 to rupture, providing access to the straw 32.

FIGS. 11 and 12 shows that the score line 50′ has been moved to a position on front of the panel 42 that is substantially adjacent to the top panel 46. It is again to be appreciated that the score line 50′ could just as easily be positioned substantially adjacent to the bottom panel 48.

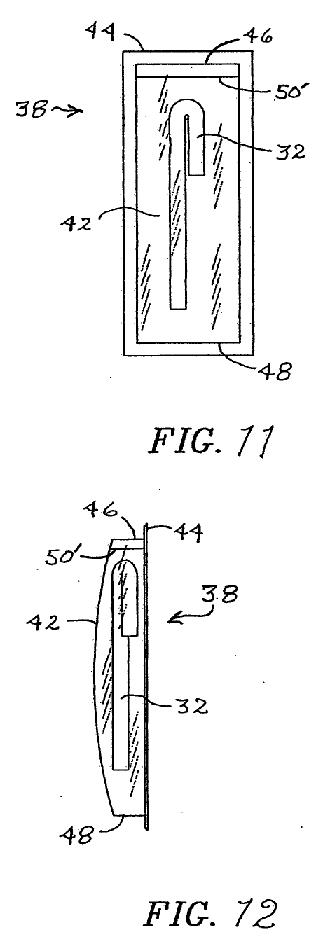

FIGS. 13 and 14 illustrate the weakened portion as 50″. As shown in FIG. 13, the score line 50″ extends only partially across the front panel 42. Nevertheless, upon the application of outside pressure, the score line 50″ will rupture, most probably with a “pop” sound.

FIGS. 15 and 16 illustrate the weakened portion as defined by a substantially V-shaped score line 62. Any application of outside pressure to the bubble package 38 will cause each side of the V-shaped score line 62 to rupture, thereby providing access to the drinking straw.

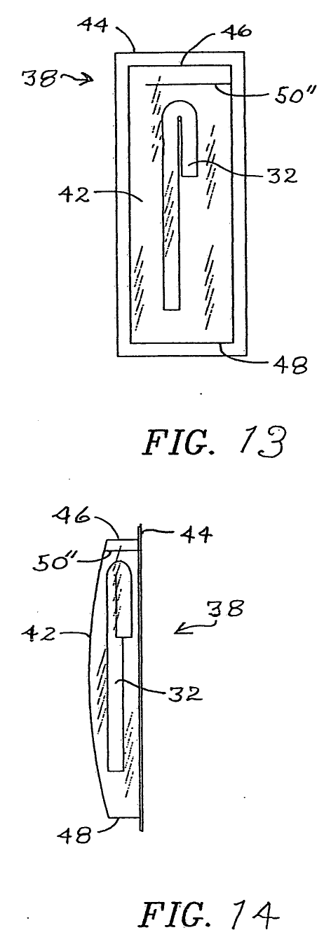

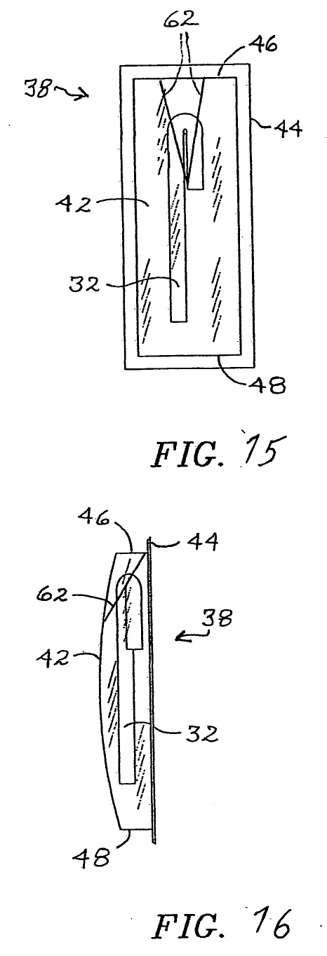

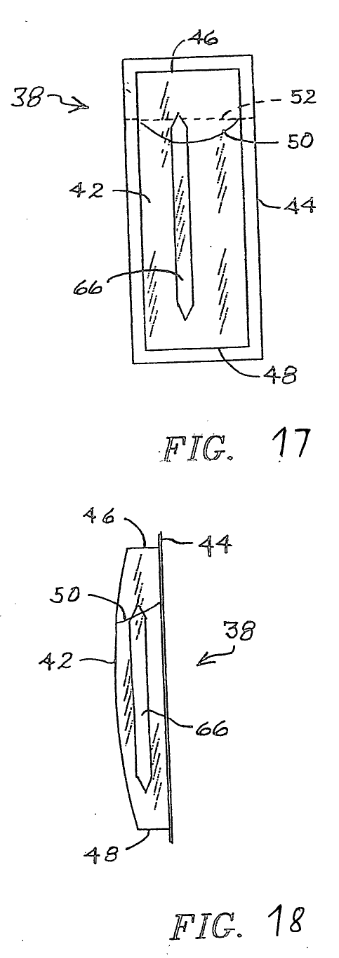

FIGS. 17 and 18 depict a rupturable bubble package 38 that is substantially identical to the embodiments of FIGS. 1-6. However, for purposes of clarity in showing that virtually any implement may be placed within the bubble package 38, the views of FIGS. 17 and 18 illustrate a toothpick within the enclosure of package 38.

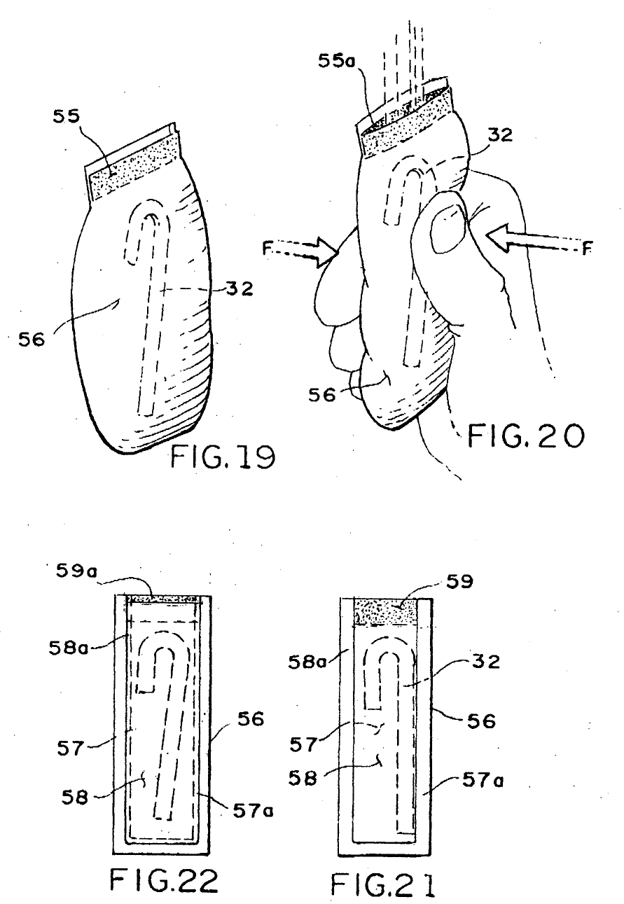

The following FIGS. 19-35 disclose a different concept of the invention in that instead of a score line being ruptured when an external pressure is applied to the package, a weakened seal will be ruptured, again, when an external pressure is applied to the bubble package, the weakened seal will rupture because of the increased air pressure within the package.

FIGS. 19 and 20 illustrate such a package. The package 56 in FIG. 19 could be made by extruding a clear plastic package as a sleeve 56, permanently seal one end of the sleeve, placing an elongated item 32 in the form of a straw into the sleeve and then seal the other end of the sleeve with a weak seal. The weak seal is established by a glue or heat adhesion. When an external pressure is applied to the package, the increased air pressure within the package will burst the weakened seal open as is shown by 55a in FIG. 20.

FIGS. 21 and 22 illustrates a different construction of the bubble package 56. In this embodiment a plastic back panel 57 is formed and a plastic bubble front panel 58 is attached to the back panel 57 and the overlapping sides are permanently sealed to each other. Between the overlaps at the top opening, a narrow weakened seal 59 is formed which will burst open once an external pressure is applied to the bubble as is shown in at 59a in FIG. 22. The overlaps of the front panel 58 and the back panel 57 are shown at 57a and 58 a which are overlaps permanently sealed to each other.

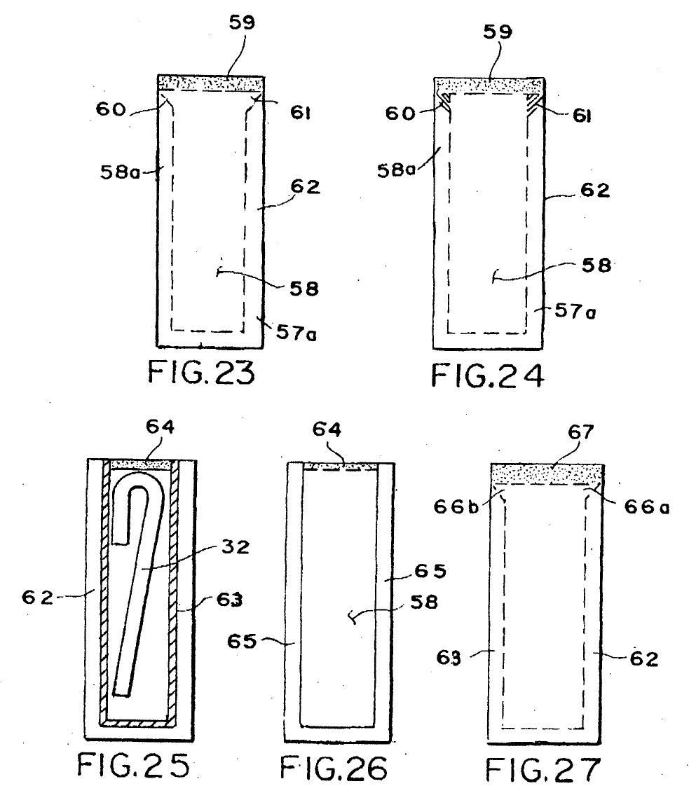

FIGS. 23 and 24 illustrate the basic construction as was shown in FIGS. 21 and 22 except that the overlaps 57a and 58a, that are permanently sealed to each other, are not completed at the upper edge of the bubble 58, whereby triangular corners 60 and 61 are left unattached. This results in that a wider weakened seal 59 can be established at this upper edge which would widen the burst opening so a wider item contained in the bubble package could be extracted there from.

FIG. 25 illustrates still another embodiment wherein two halves of bubble formations 62 and 63 are permanently sealed together to thereby form a three dimensional bubble package. A narrow and weakened seal 64 is established at the upper edge. As mentioned above, the weakened seal 64 can be accomplished by a glue or a heat adhesion. Notice the straw 32 in this package.

FIG. 26 illustrates yet another embodiment wherein a front and back bubble formations 58 are formed which are then adhered to each other at their overlaps 65 by heat adhesion or glue. A narrow, weakened seal 64 is established at the top of the package.

FIG. 27 shows the same basic construction concept of FIG. 26 but differs in that the overlaps 62 and 63 that are sealed or fastened to each other by way of a heat adhesion or glue are not quite completed at the upper edge and leaving none attached triangular section 66a and 66b. This construction allows for the establishment of a wider weakened seal 67 at the top edge.

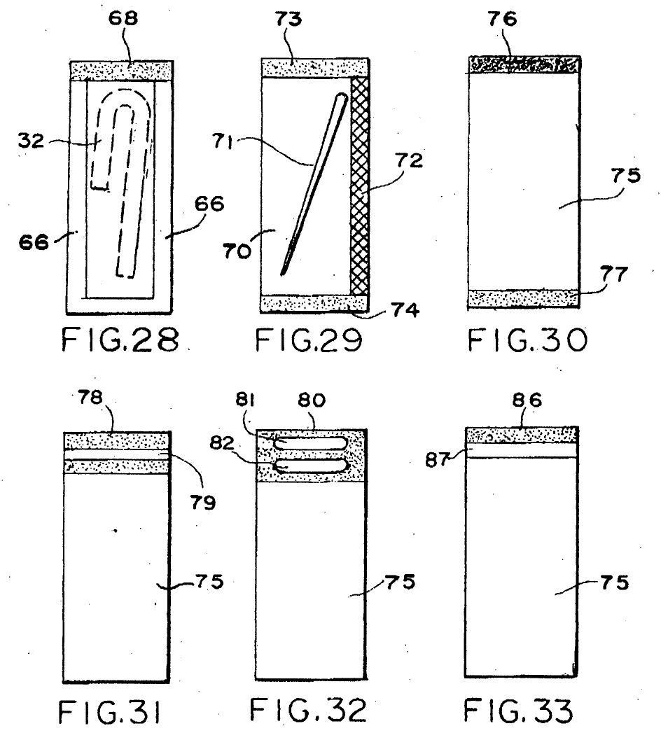

FIG. 28 differs from the bubble package shown in FIG. 19 in that the heat adhesion or glue fastened overlaps 65 and 66 stop short of the upper edge whereby a wider weakened seal 68 may be formed thereat.

FIG. 29 is a different construction altogether from previous shown packages above. This construction is made of a single plastic sheet which is formed into a tube 70 and the resulting edges of the tube 70 are heat sealed or glued together at 72. The tube may be closed at both the top and the bottom edges by way of a weakened seal 73 and 74. The user may decide which end to open depending on which end to apply the external pressure. Notice a toothpick 71 being placed into this package.

FIG. 30 shows another embodiment of construction where a sleeve or tube 75 is formed by way of an extrusion of a plastic material and both ends may be closed by different seals 76 and 77. The top seal 76 is a more solid seal which still will burst under external pressure. The bottom seal 77 is a seal that requires less strength to burst because this seal has air bubbles therein. The different seals 76 and 77 create different sounds when opened by pressure, that is, soft or loud.

FIG. 31 illustrates a different way of sealing the top edge of the package 75. This weakened seal has in its glue 78 a longitudinal air pocket formed therein. This arrangement results in a “double popper” when the seal burst when external pressure is applied to the package 75.

FIG. 32 shows a bubble package 75 that has still another novel 80 seal at its upper edge. The illustration is similar to the one shown in FIG. 31 except that the weakened seal 80 has two longitudinal air pockets formed therein. When an external pressure is applied to the bubble package 75, the resulting burst of the weakened seal 80 creates a loud pop when ruptured

FIG. 33 shows still another embodiment of the weakened seals that have elongated air pockets therein. In this embodiment there is an elongated air pocket 87 placed near the top end of the package 75 as a seam block which then is followed by a weakened seal 86.

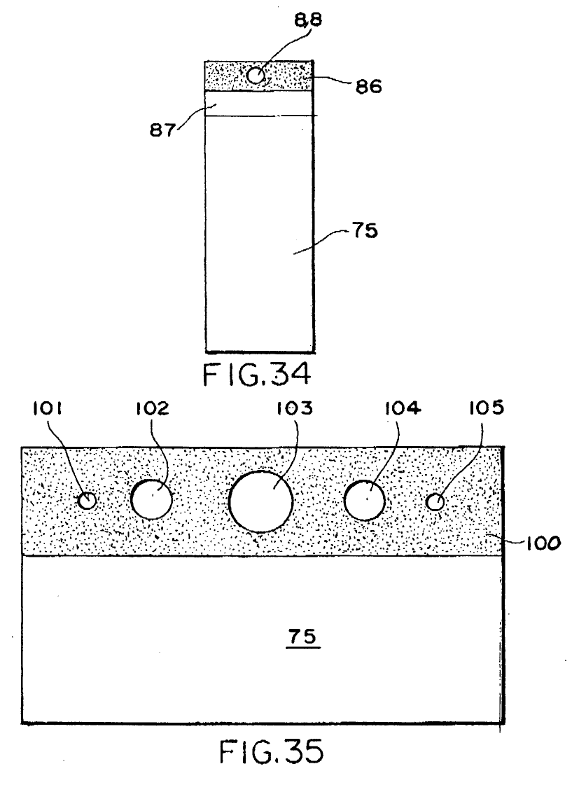

FIG. 34 illustrates another embodiment similar to FIG. 33. This embodiment still has the longitudinal and lateral air block 87 at the end of the package 75 which again is followed by and is adjacent to the weakened seal 86. However, the weakened seal 86 has a circular air pocket 88 therein which will create an extra fun “popping sound” when

FIG. 35 illustrates another embodiment which is created as an amusement article in combination with a sealed bubble package. In this embodiment the basic package is shown at 75 which has the usual weakened seal 100 at its top edge. However, the weakened seal 100 contains a multiple of differently sized air pockets 101-105 therein. The differently sized air pockets may create different sounds when they burst open. In this FIG. 35 only the top portion of the bubble package is shown. However, the remainder of the package may be filled with the requisite item and some air. It should be noted that the differently sized air pockets 101-105 may be filled with pills which can be popped through the plastic material by way of a one-sided pressure when needed. This feature is another useful inventive concept in the use of packaged sanitized items in the bubble package.

What is claim is:

1. A ruptural bubble package for containing an implement, said package comprising an enclosure formed from a flexible material, said flexible material being substantially impervious to air and fluids, whereby a positive pressure of a gaseous fluid may be maintained within said enclosure when an implement is placed therein and said bubble package is sealed, said bubble package having weakened portions thereon, said weakened portions will burst the bubble package open when an external pressure is applied to said bubble package whereby said external pressure increases the internal positive pressure of said gaseous fluid causing said weakened portion to burst.

2. The ruptural bubble package of claim 1, wherein said bubble package is constructed of a front panel, a back panel, and opposed top and bottom panels.

3. The ruptural bubble package of claim 2, wherein said weakened portion is formed on said front panel intermediate said top and bottom panels.

4. The ruptural bubble package of claim 2, wherein said weakened portion is formed on said front panel and said top panel.

5. The ruptural bubble package of claim 2, wherein said top panel is integral with said front panel and is interconnected to said back panel to define a seam.

6. The ruptural bubble package of claim 5, wherein said weakened portion is defined by said seam.

7. The ruptural bubble package of claim 2, wherein said top panel is a lip segment that extends onto said front panel, said lip segment being attached to said front panel and said weakened portion being defined by said attachment of said lip segment to said front panel.

8. The ruptural bubble package of claim 1, wherein said weakened portion comprises a score line in the surface of said bubble package.

9. The ruptural bubble package of claim 1, wherein said bubble package is constructed as a sleeve.

10. The ruptural bubble package of claim 9, wherein said sleeve has a permanent seal at one end thereof and said weakened portion is a weakened seal at another end of said sleeve.

11. The ruptural bubble package of claim 1, wherein said bubble package is constructed from two halves of plastic sheets being permanently sealed at their overlapping sides leaving a top edge free and said top edge is sealed by a weakened seal

12. The ruptural bubble package of claim 11, wherein said weakened seal extends the full extent of said top edge.

13. The ruptural bubble package of claim 11, wherein said weakened seal extends partially across said top edge.

14. The ruptural bubble package of claim 9, wherein said sleeve has a weakened seals at both ends of said sleeve.

15. The ruptural bubble package of claim 9, wherein said weakened seal has a longitudinal air pocket therein.

16. The ruptural bubble package of claim 9, wherein said weakened seal has at least two longitudinal air pockets therein.

17. The ruptural bubble package of claim 9, wherein said weakened seal has a circular air pocket centrally located therein.

18. The ruptural bubble package of claim 9, wherein said weakened seal has a multiple of differently sized air pockets therein.

19. The ruptural bubble package of claim 9, wherein said sleeve has different strengths weakened seals at each end of said sleeve.

20. The ruptural bubble package of claim 1 wherein said implement is selected from the group of items consisting of drinking straws, tooth picks, towel lets, medical instruments, pills, food and hardware items.
